(19) United States
(12) Patent Application Publication
(10) Pub. No.: US 2012/0296322 A1
(43) Pub. Date: Nov. 22, 2012

(54) LASER TREATMENT DEVICE

(75) Inventors: Iwao Yamazaki, Tokyo (JP); Akitsugu Yamazaki, Tokyo (JP)

(73) Assignee: YA-MAN LTD., Tokyo (JP)

(21) Appl. No.: 13/521,962

(22) PCT Filed: Mar. 10, 2011
(86) PCT No.: PCT/JP2011/055636

§ 371 Date: Jul. 12, 2012

(30) Foreign Application Priority Data
Mar. 15, 2010 (JP) 2010-057008

(51) Int. Cl. A61B 18/20

(52) U.S. Cl. 606/9

(57) ABSTRACT

[Problem to be Solved] It cannot be said that the irradiation range of laser light is sufficiently wide, a lot of labor is taken to irradiate the skin with laser light, and there has been a problem that efficiency of cosmetic treatment is bad.

[Solution] A laser treatment device is provided with a laser light source 40 consisting of at least one or more VCSEL array 41 that has two or more VCSEL elements 41s arranged on a single wafer and emits laser light for irradiating the skin. The laser treatment device may also be provided with: a reflected-light power detection means that detects power of reflected light of the light irradiating an irradiation part, the reflected light reflected from the irradiation part; and a control means that adjusts, in accordance with the power of the reflected light detected by the reflected-light power detection means, power of the laser light emitted from the light source means.

### TECHNICAL FIELD

The present invention relates to a laser treatment device that irradiates the skin with laser light to carry out hair removal or other cosmetic treatment.

### BACKGROUND ART

FIG. 13 shows the interior of a conventional laser treatment device in a manner that part thereof is cut away.

This conventional device is disclosed in Patent Literature 1.

In FIG. 13, the laser treatment device is denoted by 101, an outer case is denoted by 102, a gripping unit is denoted by 103, a head unit is denoted by 104, an opening of the head unit 104 is denoted by 104A, and an operation panel unit is denoted by 105.

An optical unit is denoted by 106, a semiconductor laser which emits laser light is denoted by 107, a spherical lens which condenses the laser light thereof is denoted by 108, and a heatsink which dissipates the heat of the semiconductor laser 107 is denoted by 109.

Furthermore, a vibration motor fixed to the optical unit 106 is denoted by 110, a motor shaft of the vibration motor 110 is denoted by 111, an eccentric weight fixed to the motor shaft 111 is denoted by 112, a pivot point of vibrations is denoted by 113, and an irradiation surface to be subjected to cosmetic treatment is denoted by 114.

Next, operation will be explained.

The laser light emitted from the semiconductor laser 107 is condensed by the spherical lens 108 and irradiates the irradiation surface 114. In the case of this irradiation at one point, the irradiation range on the irradiation surface 114 has a diameter of about 2 to 3 mm.

### CITATION LIST

### Patent Literature

Patent Literature 1: Japanese Patent Application Laid-Open No. 2003-135484

### SUMMARY OF INVENTION

### Technical Problems

The conventional laser treatment device has the above described configuration. Therefore, it cannot be said that the irradiation range of the laser light is sufficiently large, and the laser light has to be moved back and forth with respect to the skin when a large area is to be irradiated with laser light, and, therefore, there has been a problem that labor is required and efficiency is bad.

The cause thereof is that an output peak is on an optical axis since the semiconductor laser 107 used as a laser light source is a one-point light-emission type, and the laser light of this one-point irradiation inevitably has a limit on the irradiation range.

If a plurality of semiconductor lasers of the one-point light-emission type are provided in order to improve this, laser light irradiation of a wide range is tentatively enabled. However, a mounting volume corresponding to the number of the semiconductor lasers is required, and peripheral constituent elements such as heatsinks accompanying them are increased at the same time. Therefore, the overall device becomes larger and not practical.

This invention has been accomplished in order to solve above described problems, and it is an object to improve the efficiency of cosmetic treatment by irradiating the skin with high-power wide-range laser light while suppressing increase in the size thereof to ensure practicality.

### Solution to Problem

A laser treatment device according to claim 1 is configured to be provided with: a surface-emitting laser array as alight source means, the surface-emitting laser array that has two or more laser elements arranged on a single wafer and emits laser light for irradiating an irradiation part.

In the laser treatment device according to claim 2, the surface-emitting laser array according to claim 1 is a vertical cavity surface emitting laser array that includes two or more vertical cavity surface emitting laser elements arranged on the single wafer.

In the laser treatment device according to claim 3, the light source means according to claim 1 is provided with a light guide means that receives the emitted laser light and guides the light to the irradiation part.

In the laser treatment device according to claim 4, the light source means according to claim 1 is provided with a battery that drives the surface-emitting laser array.

In the laser treatment device according to claim 5, the light source means according to claim 1 is provided with the two surface-emitting laser arrays connected in series.

In the laser treatment device according to claim 6, the light source means according to claim 1 is provided with a light diffusion means that diffuses the laser light for irradiating the irradiation part toward the irradiation part.

The laser treatment device according to claim 7 is provided with a reflected-light power detection means that detects power of reflected light from the irradiation part; and a control means that adjusts, in accordance with the reflected light power detected by the reflected-light power detection means, the power of the laser light.

In the laser treatment device according to claim 8, the light source means according to claim 1 is provided with a contact detection means that detects contact with the irradiation part to be irradiated with the laser light; and a control means that causes the light source means to emit laser light only while the contact detection means is detecting the contact with the irradiation part.

In the laser treatment device according to claim 9, after the irradiation part is irradiated with the laser light for predetermined time, the control means according to claim 8 causes the light source means to stop emission of the laser light.

### Advantageous Effects of Invention

As described above, according to the laser treatment device according to claim 1, the device is configured to be provided with the at least one or more surface-emitting laser array as the light source means, the surface-emitting laser array that has the two or more laser elements arranged on the single wafer and emits laser light for irradiating the irradiation part. Therefore, high-power and wide-range laser light having the synthetic intensity distribution, which is the synthesis of the intensity distribution of the laser light from the laser elements, is emitted from the light source means. This high-power and wide-range laser light irradiates the irradiation part, and an effect that the efficiency of cosmetic treatment can be improved is obtained.

According to the laser treatment device according to claim 2, the light source means is configured to be provided with the vertical cavity surface emitting laser array that includes the two or more vertical cavity surface emitting laser elements arranged on the single wafer. Therefore, since the vertical cavity surface emitting laser elements are used, an effect that the elements can be easily formed into an array is obtained.

According to the laser treatment device according to claim 3, the light source means is configured to be provided with the light guide means that receives the emitted laser light and guides the light to the irradiation part. Therefore, an effect that the laser light emitted by the light source means can be easily guided to the irradiation part is obtained.

According to the laser treatment device according to claim 4, the device is configured to be provided with the battery that drives the surface-emitting laser array. Therefore, a large AC adapter or the like is not required to be used as a drive circuit of the surface-emitting laser array, which requires a large current, increase in the size of the laser treatment device using the surface-emitting array can be suppressed, and an effect that practicality can be ensured is obtained.

According to the laser treatment device according to claim 5, the light source means is configured to be provided with the two surface-emitting laser arrays connected in series. Therefore, an effect that the most-efficient laser treatment device can be constituted by using four 1.2-V batteries is obtained.

According to the laser treatment device according to claim 6, the device is configured to be provided with the light diffusion means that diffuses the laser light for irradiating the irradiation part toward the irradiation part. Therefore, the laser light is diffused and radiated to the irradiation part, and an effect that the user can be protected from a trouble such as burn can be obtained. Moreover, the high-power and wide-range laser light can be distributed to the skin over a further wider area, and an effect that the efficiency of cosmetic treatment can be improved is obtained. Furthermore, the irradiation part can be irradiated with the laser light having high uniformity, and an effect that irradiation unevenness of cosmetic treatment can be reduced is obtained.

According to the laser treatment device according to claim 7, the device is configured to be provided with the reflected-light power detection means that detects power of reflected light of the light irradiating the irradiation part, the reflected light reflected from the irradiation part; and the control means that adjusts, in accordance with the reflected light power detected by the reflected-light power detection means, the power of the laser light emitted from the light source means.

Therefore, the power of the laser light radiated to the irradiation part can be optimized in accordance with the individual differences in the color of the irradiation part, an effect that safety of the high-power laser treatment device can be ensured is obtained.

According to the laser treatment device according to claim 8, the device is configured to be provided with the contact detection means that detects contact with the irradiation part to be irradiated with the laser light; and the control means that causes the light source means to emit laser light only while the contact detection means is detecting the contact with the irradiation part. Therefore, as long as contact with the irradiation part to be irradiated with the laser light is not detected by the contact detection means, the laser light source does not emit laser light, an unexpected trouble that, for example, eyes are erroneously irradiated with laser light can be prevented, and an effect that safety of the high-power laser treatment device can be ensured is obtained.

According to the laser treatment device according to claim 9, after the irradiation part is irradiated with the laser light for predetermined time, the control means is configured to cause the light source means to stop emission of the laser light. Therefore, the trouble that laser light is excessively radiated to the irradiation part of the single location can be prevented, and an effect that safety of the high-power laser treatment device can be further ensured is obtained.

### BRIEF DESCRIPTION OF DRAWINGS

FIG. 1 shows drawings showing a constitution of a laser treatment device according to a first embodiment of this invention.

FIG. 2 shows drawings showing a constitution of a laser light source of FIG. 1.

FIG. 3 is a drawing for explaining design examples of the case in which a battery(ies) is used as a drive power source of the laser light source.

FIG. 4 shows drawings showing a constitution of a laser treatment device according to a second embodiment of this invention.

FIG. 5 shows drawings showing a constitution of a laser treatment device according to a third embodiment of this invention.

FIG. 6 is a block diagram showing a circuit configuration of the laser treatment device according to the third embodiment of this invention.

FIG. 7 is a flow chart showing operation of the laser treatment device according to the third embodiment of this invention.

FIG. 8 shows drawings showing a constitution of a laser treatment device according to a fourth embodiment of this invention.

FIG. 9 is a block diagram showing a circuit configuration of the laser treatment device according to the fourth embodiment of this invention.

FIG. 10 is a flow chart showing operation of the laser treatment device according to the fourth embodiment of this invention.

FIG. 11 is a flow chart showing operation of the laser treatment device according to the fourth embodiment of this invention.

FIG. 12 shows timing charts for explaining operation of the treatment device of FIG. 11.

FIG. 13 is a drawing showing the configuration of a conventional laser treatment device.

### DESCRIPTION OF EMBODIMENTS

Hereinafter, embodiments of this invention will be explained in detail with reference to drawings. In the drawings, the same constitutions or corresponding constitutions are denoted by the same symbols.

### First Embodiment

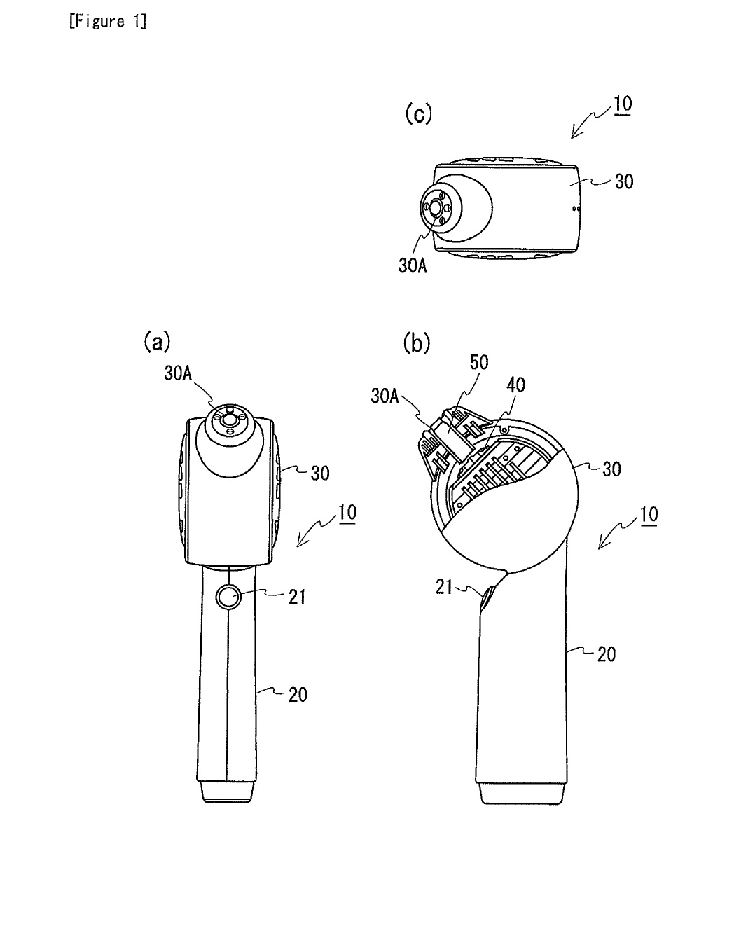

FIG. 1 shows drawings showing the constitution of a laser treatment device according to a first embodiment of this invention. FIG. 1 (a), FIG. 1 (b), and FIG. 1 (c) are a front view, a lateral view, and a top view of the laser treatment device, respectively. Particularly, FIG. 1 (b) shows an internal structure of the laser treatment device in a manner that part thereof is cut away.

In FIG. 1, the laser treatment device is denoted by 10, a gripping unit gripped by a user of the laser treatment device 10 is denoted by 20, an irradiation button for irradiating the laser light is denoted by 21, a head unit in which a laser light source and other optical devices are built in is denoted by 30, an opening of the head unit 30 is denoted by 30A, a laser light source (light source means) is denoted by 40, and a light guide (light guiding means) which receives laser light emitted from the laser light source 40 to irradiate the skin therewith and is made of, for example, polymethylmethacrylate is denoted by 50.

The laser light source 40 is a constituent element that characterizes the invention of the present application. The laser light source 40 is provided with a VCSEL array in which a plurality of VCSEL (*1) elements are arranged on a single wafer, wherein the elements resonate light in a direction perpendicular to a board surface and emit the light in the direction.

(*1) “VCSEL” . . . An abbreviation for Vertical Cavity Surface Emitting Laser. In the present description, abbreviated as “VCSEL”.

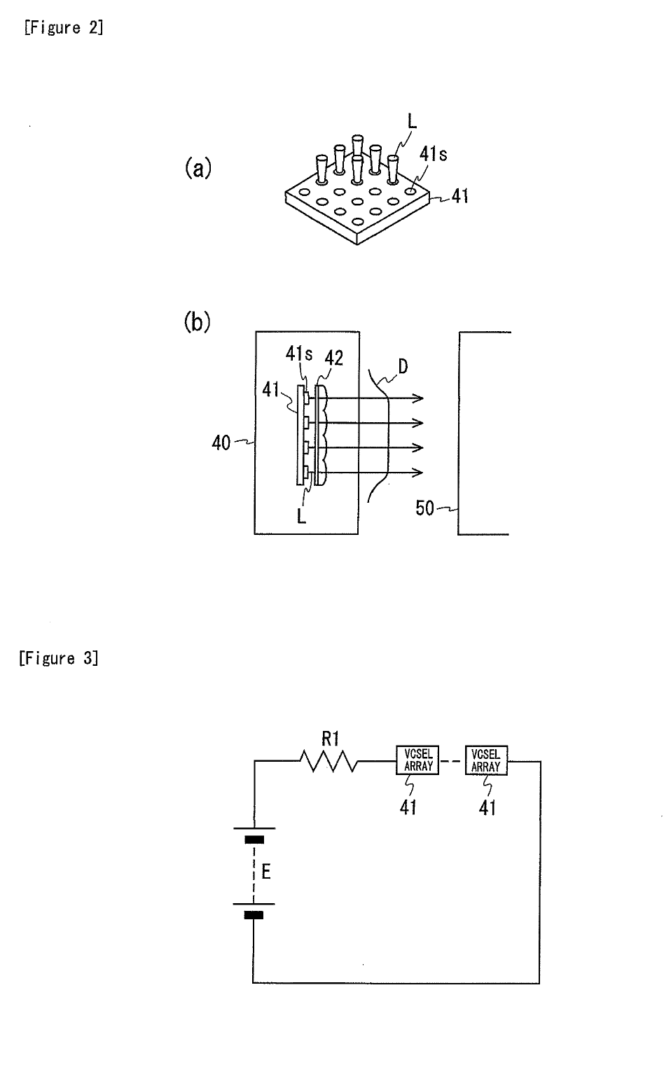

FIG. 2 shows drawings showing a constitution of the laser light source of FIG. 1. FIG. 2 (a) is a perspective view of the VCSEL array provided in the laser light source 40, and FIG. 2 (b) schematically shows a lateral view of the laser light source 40, which emits laser light to the light guide 50.

In FIG. 2, the VCSEL array provided in the laser light source 40 is denoted by 41, a plurality of VCSEL elements constituting the VCSEL array 41 are denoted by 41s, laser light emitted from the VCSEL elements 41s is denoted by L, a microlens array which collimates the laser light L is denoted by 42, and virtually-illustrated synthesis intensity distribution of the laser light L is denoted by D.

As is generally well known, the VCSEL elements 41s have advantages, for example, that the elements can be easily formed into an array when compared with a laser element of an end-face light-emitting type which has a resonator in parallel to a board surface and emits light from a cleaved lateral surface. When the plurality of VCSEL elements 41s are densely arranged on the single wafer to constitute the VCSEL array 41 as shown in FIG. 2 (a), the laser light which has a synthesis of the intensity of the laser light L thereof is emitted from the entire emitting surface of the VCSEL array 41, and laser light having high power and a wide range is realized.

In the laser treatment device 10 using the VCSEL array 41 like this, when a user turns on the irradiation button 21, as shown in FIG. 2 (b), the laser light L emitted from the VCSEL array 41 is emitted through the microlens array 42 and from the laser light source 40 as laser light having the synthetic intensity distribution D. The laser light having this synthetic intensity distribution D transmits through the light guide 50 and is radiated from the opening 30A to the skin of the user serving as an irradiation part. Since the laser light having the synthetic intensity distribution D of which power and range are increased by the VCSEL array 41 is radiated, the efficiency of hair removal or other cosmetic treatment can be improved.

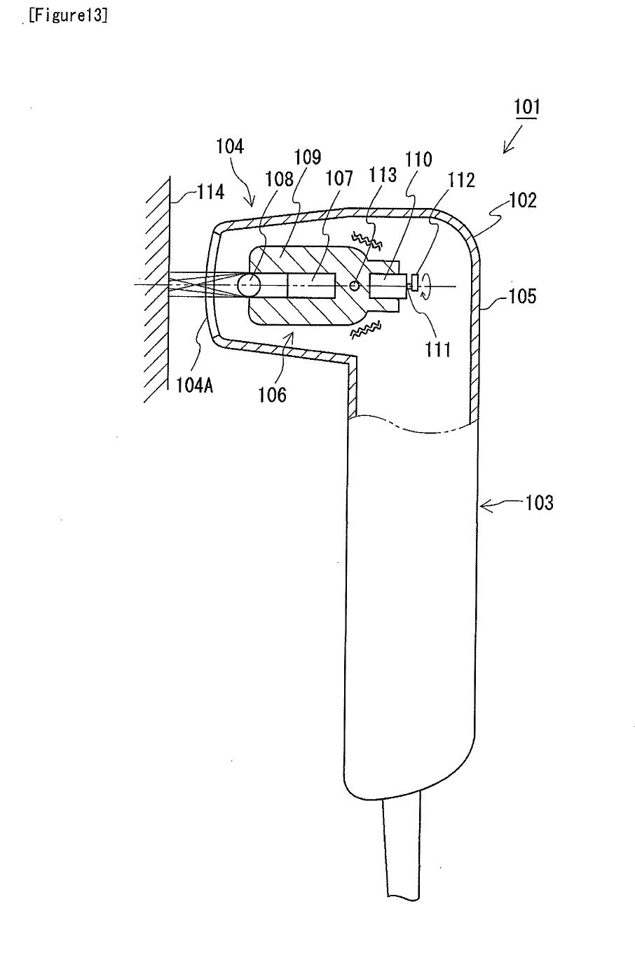

According to the laser treatment device 10 experimentally produced by the inventor of the present application, the laser light can be radiated across a wide range having a diameter of about 20 mm. In consideration of the fact that the irradiation range of the case of the one-point irradiation by the conventional laser treatment device 101 of FIG. 13 has been about a diameter of about 2 to 3 mm, wide-range irradiation of about 100 times can be carried out when converted by an area ratio. The irradiation range can be further widened by increasing the number of the VCSEL array 41.

Since the VCSEL elements 41s are arranged on the single wafer, the mounting volume thereof occupied in the laser treatment device 10 is not increased much, and not many peripheral constituent elements such as heatsinks are required. However, compared with the conventional laser treatment device 101, a considerably large current has to be supplied to the VCSEL array 41. An AC adapter which supplies such a large current is not commercially available, the size of a circuit is increased if the required large current is to be generated from an alternating current of 100 V, and the laser treatment device 10 becomes impractical.

Therefore, in the first embodiment, a battery(ies) is used as a drive power source of the laser light source 40 so that the laser power source 40 provided with the VCSEL array 41 can be driven even for a short period of time. In this manner, the increased power and widened area of laser light are realized, at the same time, increase in the size of circuit is suppressed, and practicality of the laser treatment device 10 is ensured.

FIG. 3 is a drawing for explaining design examples of the case in which the battery(ies) is used as the drive power source of the laser light source 40.

Herein, rechargeable batteries of 1.2 V are connected in series and used as the drive power source, the drive voltage per each VCSEL array 41 is 2 V, the efficiency of transformation is higher in step-down than in step-up, and the voltage is decreased by a resistance R1 to drive the VCSEL array 41. The drive voltage of other devices provided in the laser treatment device 10 is 3 V.

Design Example (a) . . . When the number of the rechargeable battery is one, a battery voltage E becomes 1.2 V. Therefore, the number of the drivable VCSEL array 41 is 0, and the difference between the drive voltage of the other devices and the battery voltage E is +1.8 V.

Design Example (b) . . . When the number of the rechargeable batteries is two, the battery voltage E becomes 2.4 V. Therefore, the number of the drivable VCSEL array 41 is one, and the step-down voltage caused by the resistance R1 in this case is “2.4 V−2.0 V=0.4V”. The difference between the drive voltage of the other devices and the battery voltage E is “3.0 V−2.4 V=+0.6 V”.

Design Example (c) . . . When the number of the rechargeable batteries is three, the battery voltage E becomes 3.6 V. Therefore, the number of the drivable VCSEL array 41 is one, and the step-down voltage caused by the resistance R1 in this case is “3.6V−2.0V=1.6V”. The difference between the drive voltage of the other devices and the battery voltage E is “3.0 V−3.6 V=−0.6 V”.

Design Example (d) . . . When the number of the rechargeable batteries is four, the battery voltage E becomes 4.8 V. Therefore, the number of the drivable VCSEL arrays 41 is increased to two, and the step-down voltage caused by the resistance R1 in this case is “4.8 V−2×2.0 V=0.8 V”. The difference between the drive voltage of the other devices and the battery voltage E is “3.0 V−4.8 V=−1.8 V”.

Design Example (e) . . . When the number of the rechargeable batteries is five, the battery voltage E becomes 6.0 V. Therefore, the number of the drivable VCSEL array 41 is increased to three, and the step-down voltage caused by the resistance R1 in this case is “6.0 V−3×2.0 V=0.0 V”. The difference between the drive voltage of the other devices and the battery voltage E is “3.0 V−6.0 V=−3.0 V”.

As described above, the cases of the Design Examples (b) and (d) in which the number of the rechargeable batteries with respect to one VCSEL array 41 is two are more efficient than Design Example (a) in which the VCSEL array 41 cannot be driven and Design Example (c) in which one VCSEL array 41 is driven by using three rechargeable batteries. However, Design Example (b) requires step-up of 0.6 V in order to drive the other devices; and, when it is taken into consideration that the efficiency of transformation in step-up is lower than that of step-down, it can be understood that Design Example (d) in which the other devices can be driven by the step-down of 1.8 V is the most efficient. In Design Example (e), the three VCSEL arrays 41 can be driven with the five rechargeable batteries, efficiency is good with a step-down voltage of 0.0 V; however, since this example cannot be driven when the battery voltage E drops due to usage, this example is not employed in consideration of the viewpoint of design margins, etc.

The arrangement of the VCSEL elements 41s constituting the VCSEL array 41 may be one-dimensional linear arrangement or two-dimensional planar arrangement.

The values of N and M can be changed in accordance with design specifications in the case of the one-dimensional arrangement in which the VCSEL elements 41s corresponding to N are arranged or in the case of the two-dimensional arrangement in which the elements are arranged in a matrix of N×M. Furthermore, the shape of the two-dimensional arrangement is not limited to the matrix of N×M, and various patterns such as hexagonal arrangement can be employed.

Furthermore, the number of the VCSEL arrays 41 used in the laser light source 40 is not limited to one or two, but may be three or more; and the manner of connecting the VCSEL arrays 41 is not limited to serial connection, but may be parallel connection.

Furthermore, the surface-emitting laser array used in the laser light source 40 is not limited to the VCSEL array 41 using the VCSEL elements 41s. As long as at least one or more surface-emitting laser array in which two or more laser elements are arranged on a single wafer is used as the laser light source 40, this first embodiment can be implemented.

As described above, according to the first embodiment, the two or more VCSEL elements 41s are arranged on the single wafer so that at least one or more VCSEL array 41, which emits laser light for irradiating the skin, is provided as the laser light source 40. Therefore, high-power and wide-range laser light having the synthetic intensity distribution D, which is the synthesis of the intensity distribution of the laser light L from the VCSEL elements 41s, is emitted from the laser light source 40. This high-power and wide-range laser light irradiates the skin, and an effect that the efficiency of cosmetic treatment can be improved is obtained. Since the VCSEL elements 41s are used, an effect that the elements can be easily formed into an array is obtained.

Moreover, according to the first embodiment, the light guide 50, which receives the laser light emitted from the laser light source 40 and guides the light to the skin, is provided; therefore, an effect that the laser light emitted by the laser light source 40 can be easily guided to the skin is obtained.

Furthermore, according to the first embodiment, the batteries, which drive the VCSEL arrays 41, are provided. Therefore, a large AC adapter or the like is not required to be used as a drive circuit of the VCSEL arrays 41, which require a large current, increase in the size of circuit can be suppressed, and an effect that practicality of the laser treatment device 10 using the VCSEL arrays 41 can be ensured is obtained.

Furthermore, according to the first embodiment, the laser light source 40 is provided with the two VCSEL arrays 41 connected in series. Therefore, an effect that the most-efficient laser treatment device 10 can be constituted by using four 1.2-V batteries is obtained.

### Second Embodiment

As described in the first embodiment, the laser treatment device 10 uses the VCSEL array 41 in the laser light source 40. Therefore, the laser light radiated from the laser treatment device 10 has high power, and there is a risk that, if erroneously used, a trouble such as burn can be caused. In below second to fourth embodiments, safety measures about the power-increased laser light will be explained.

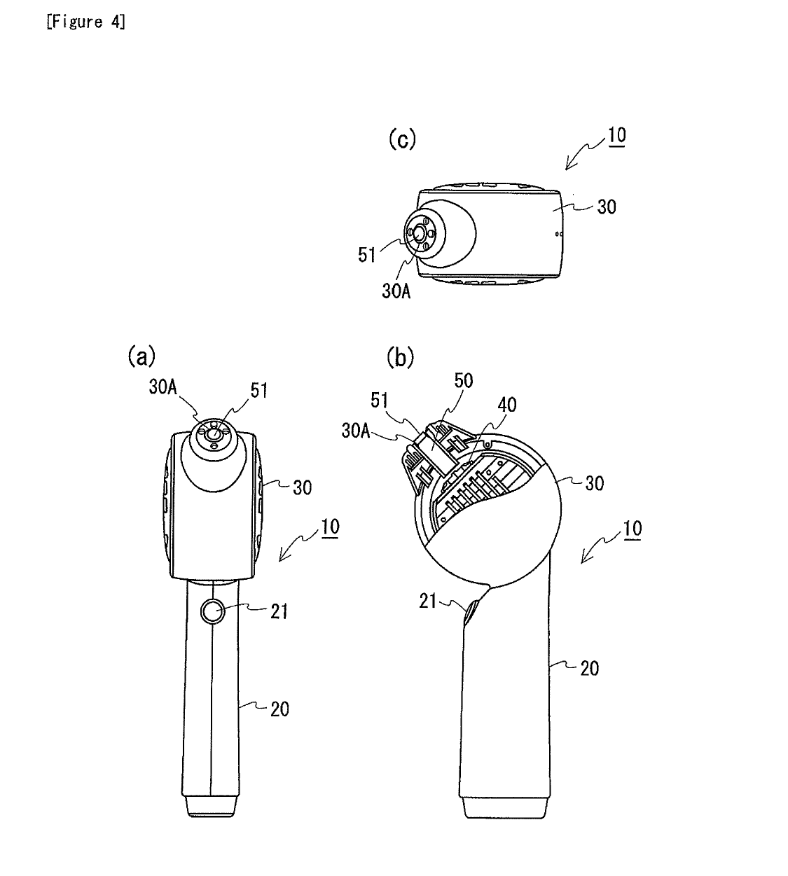

FIG. 4 shows drawings showing a constitution of a laser treatment device according to the second embodiment of this invention.

In FIG. 4, a light diffusion plate (light diffusion means) provided at the opening 30A is denoted by 51. The light diffusion plate 51 works to diffuse, toward the skin, the laser light emitted from the light guide 50.

In FIG. 4, the light diffusion plate 51 is provided at the opening 30A in a light emitting side of the light guide 50, and the laser light emitted from the light guide 50 is radiated so as to be diffused toward the skin of a predetermined range by the light diffusion plate 51.

When such a configuration is employed, a situation that high-power laser light intensively irradiates a narrow range of the skin can be prevented, and the user can be protected from a trouble such as burn. In addition to that, by virtue of the light diffusion plate 51, the high-power and wide-range laser light can be distributed to the skin over a further wide range, efficiency of cosmetic treatment can be improved, high uniformity can be imparted to the laser light, and irradiation unevenness of cosmetic treatment can be reduced.

As described above, according to the second embodiment, the light diffusion plate 51 which diffuses the laser light, which has been emitted from the light guide 50, toward the skin is provided. Therefore, the laser light is diffused and radiated to the skin, and an effect that the user can be protected from a trouble such as burn can be obtained. Moreover, the high-power and wide-range laser light can be distributed to the skin over a further wider area, and an effect that the efficiency of cosmetic treatment can be improved is obtained. Furthermore, the skin can be irradiated with the laser light having high uniformity, and an effect that irradiation unevenness of cosmetic treatment can be reduced is obtained.

### Third Embodiment

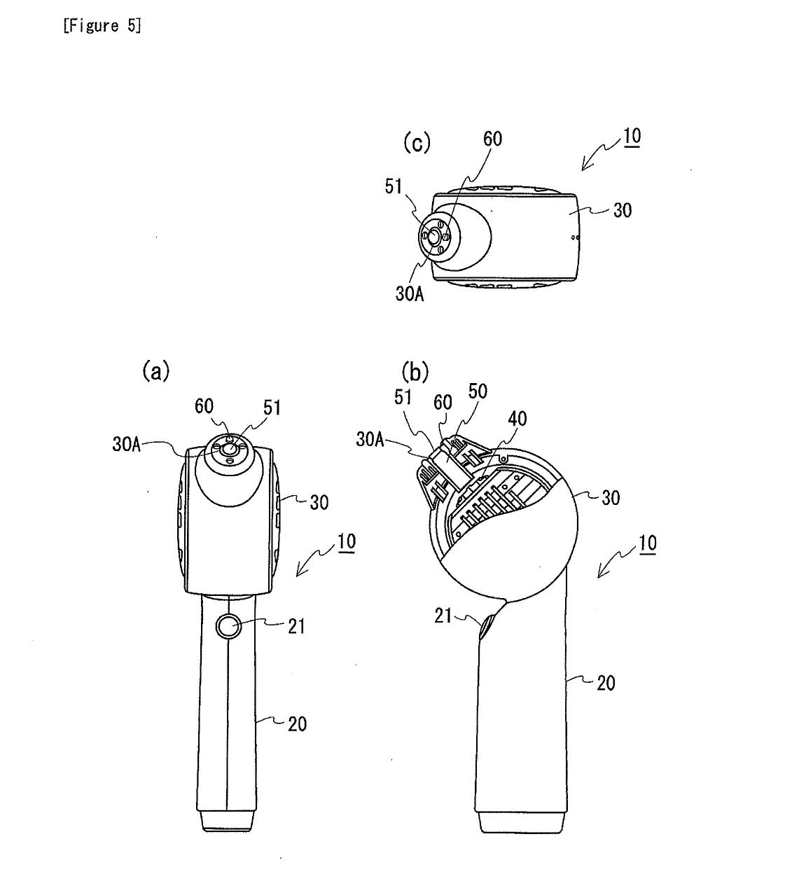

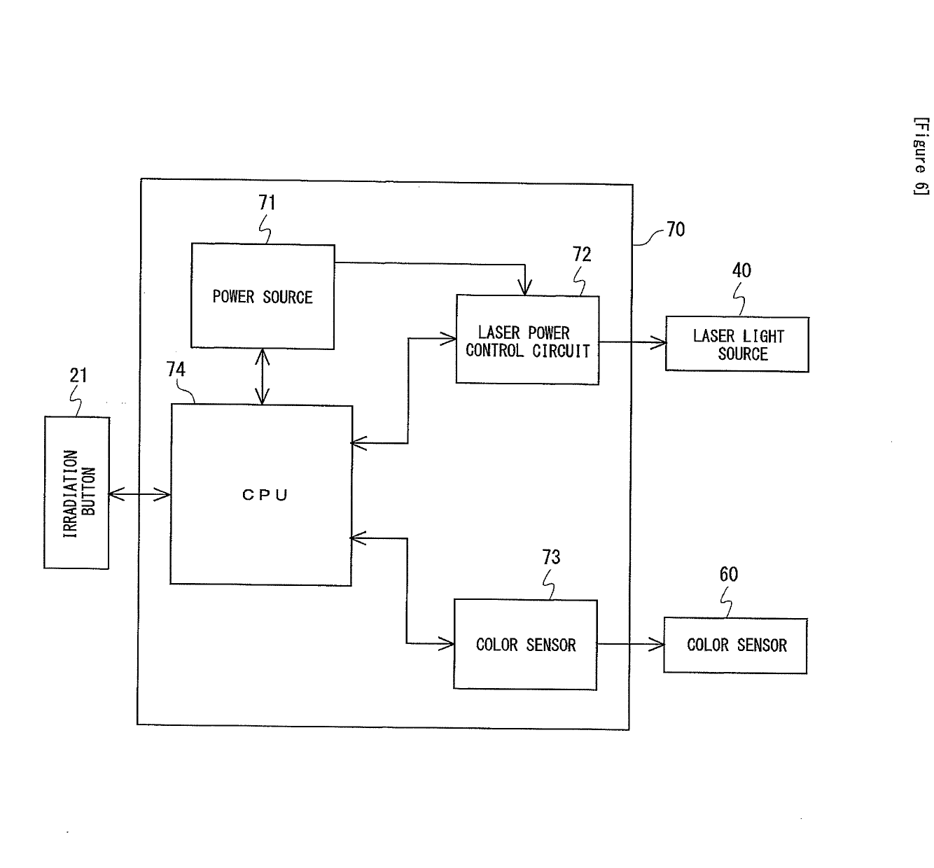

FIG. 5 shows drawings showing a constitution of a laser treatment device according to the third embodiment of this invention.

In FIG. 5, a color sensor (reflected-light power detection means) provided in the periphery of the opening 30A is denoted by 60. The color sensor 60 works to detect the power of reflected light of the laser light radiated from the laser treatment device 10 to the skin, wherein the reflected light is reflected from the skin.

FIG. 6 is a block diagram showing a circuit configuration of the laser treatment device according to the third embodiment of this invention.

In FIG. 6, a circuit is denoted by 70, a power source is denoted by 71, a laser-power control circuit (light source means) is denoted by 72, a color sensor processing circuit (reflected-light power detection means) is denoted by 73, and a CPU (control means) is denoted by 74.

Next, operation will be explained.

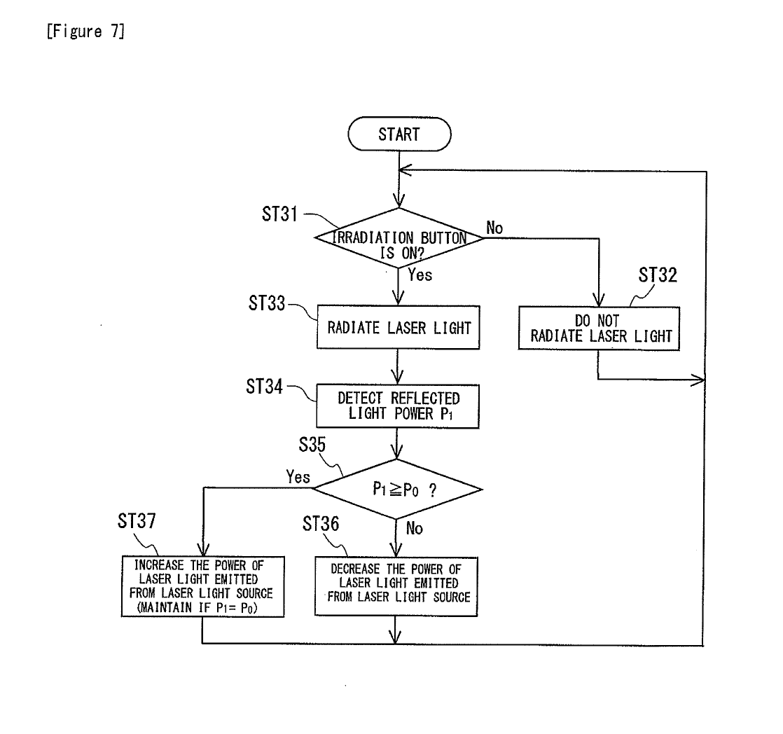

FIG. 7 is a flow chart showing the operation of the laser treatment device according to the third embodiment of this invention.

When the power source 71 is turned on to activate the laser treatment device 10, first, the CPU 74 determines whether the irradiation button 21 has been turned on or not (step ST31). While the irradiation button 21 has not been turned on (NO in step ST31), the CPU 74 does not cause the laser light to be emitted from the laser light source 40 (step ST32), and a standby state is obtained (NO in step ST31 and step ST32).

When the irradiation button 21 is turned on in the standby state (YES in step ST31), the CPU 74 controls the laser power control circuit 72 and causes the laser light source 40 to emit laser light (stepST33). The laser light is radiated to the skin via the light guide 50 and the light diffusion plate 51.

Upon this radiation, in consideration of the fact that the rate of absorption of laser light is varied depending on the individual differences in the color of the skin, the CPU 74 carries out below control. The laser light reflected by the skin and returned is received by the color sensor 60 and detected as reflected light power P1 by the color sensor processing circuit 73 (step ST34). Then, the CPU 74 checks the reflected light power P1 and optimizes the power of the laser light radiated to the skin.

Specifically, for example, if the reflected light power P1 is lower than predetermined recommended light power P0 (NO in step ST35), it is determined that the rate of the laser light absorbed by the skin is high, and the CPU 74 controls the laser power control circuit 72 and reduces the power of the laser light emitted from the laser light source 40 in order to reduce the influence of the high-power laser light exerted on the skin (step ST36).

On the other hand, for example, if the reflected light power P1 is higher than the predetermined recommended power P0 (YES in step ST35), it is determined that the rate of the laser light absorbed by the skin is low, and the CPU 74 controls the laser power control circuit 72 and increases the power of the laser light emitted from the laser light source 40 in order to improve treatment efficiency (step ST37).

For example, if the reflected light power P1 is equal to the predetermined recommended light power P0 (YES in step ST35), it is determined that the rate of the laser light absorbed by the skin is appropriate, and the CPU 74 maintains the power of the laser light emitted from the laser light source 40 (described in parentheses in step ST37).

When such a configuration is employed, the power of the laser light radiated to the skin can be optimized in accordance with the individual differences in the color of the skin, safety of the high-power laser treatment device 10 can be ensured, and treatment efficiency can be improved.

In optimization of the power of the laser light, the CPU 74 may control the laser power control circuit 72 so as to adjust the power level per se or the duty rate of the light pulses of the laser light emitted from the laser light source 40. Also, for example, an optical attenuator or the like may be provided in the emitting side of the laser light source 40 so that the optical attenuator is adjusted by the CPU 74.

The light received by the color sensor 60 is not limited to the laser light emitted from the laser light source 40. Light other than the laser light of the laser light source 40 may be radiated to the skin, and the reflected light thereof may be received by the color sensor 60. However, when the power of the reflected light, from the skin, of the laser light of the laser light source 40 is detected by the color sensor 60, the reflected light from the skin can be easily generated, and optimization of the power of the laser light is facilitated.

As described above, according to the third embodiment, the device is configured to be provided with: the color sensor 60 and the color sensor processing circuit 73, which detect the reflected light power P1, from the skin, of the laser light radiated to the skin in step ST34; and the CPU 74, which controls the laser power control circuit 72 and adjusts the power of the laser light emitted from the laser light source 40 in step ST36 or in step ST37 in accordance with the result of the comparison in step ST35 between the reflected power P1 detected by the color sensor 60 and the color sensor processing circuit 73 and the predetermined recommended light power P0. Therefore, the power of the radiated laser light can be optimized in accordance with the individual differences in the color of the skin, an effect that safety of the high-power laser treatment device can be ensured is obtained, an effect that treatment efficiency can be improved is obtained, the reflected light from the skin can be easily generated by detecting the reflected light power P1 of the laser light from the skin, and an effect that optimization of the power of the laser light is facilitated is obtained.

### Fourth Embodiment

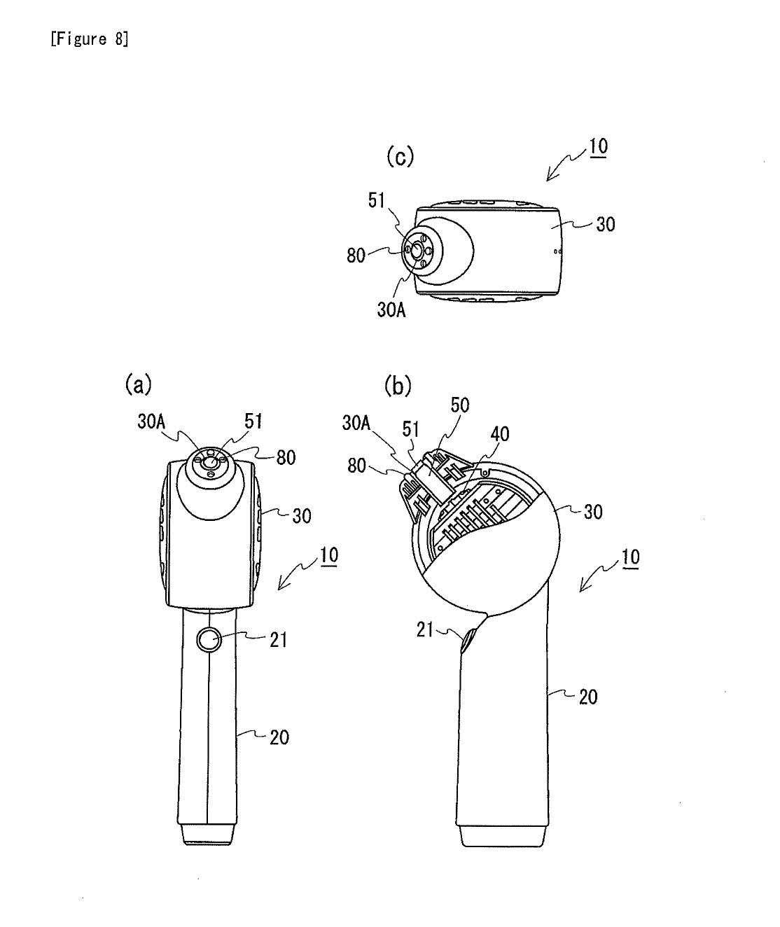

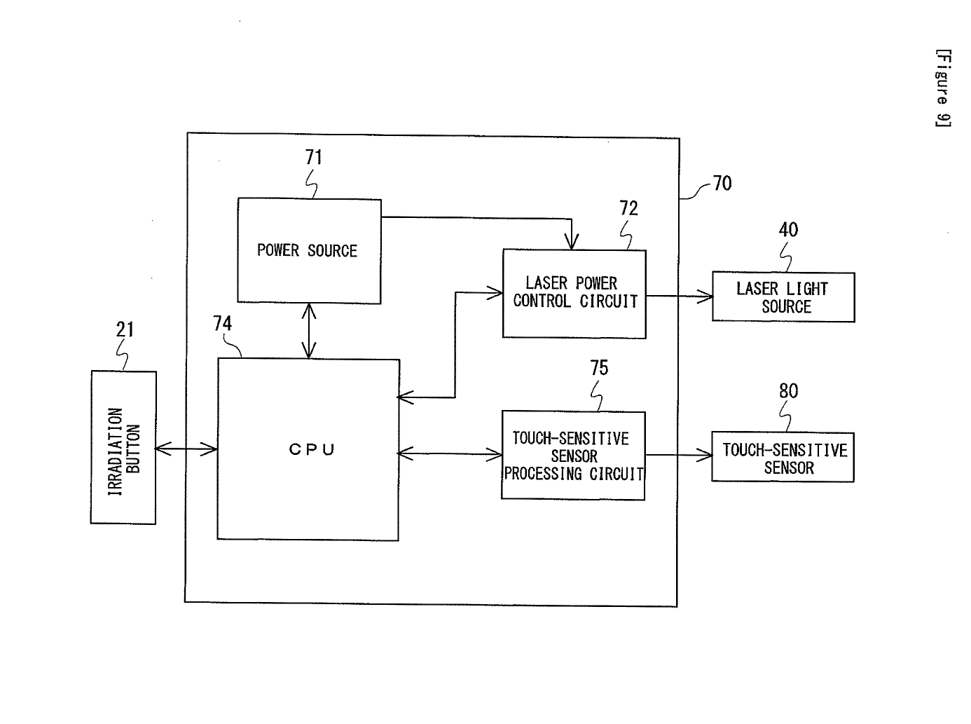

FIG. 8 shows drawings showing a constitution of a laser treatment device according to the fourth embodiment of this invention.

In FIG. 8, a touch-sensitive sensor (contact detection means) provided in the periphery of the opening 30A is denoted by 80. The touch-sensitive sensor 80 works to detect the contact with the skin irradiated with the laser light.

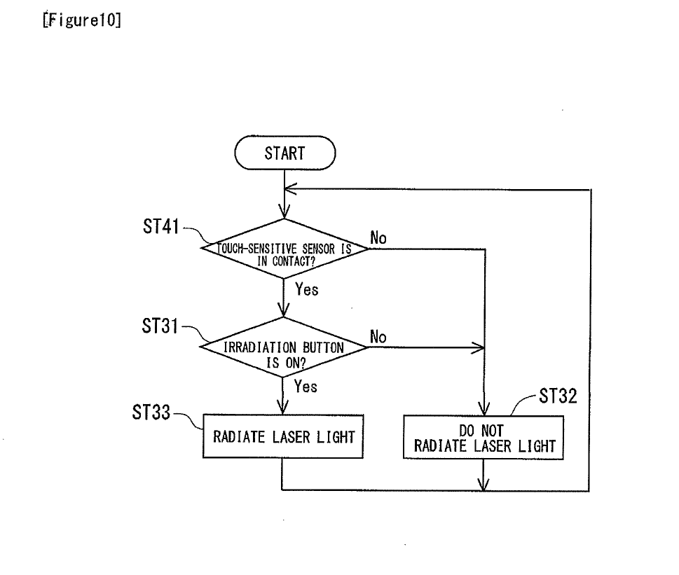

FIG. 9 is a block diagram showing a circuit configuration of the laser treatment device according to the fourth embodiment of this invention.

In FIG. 9, a touch-sensitive sensor processing circuit (contact detection means) is denoted by 75.

Next, operation will be explained.

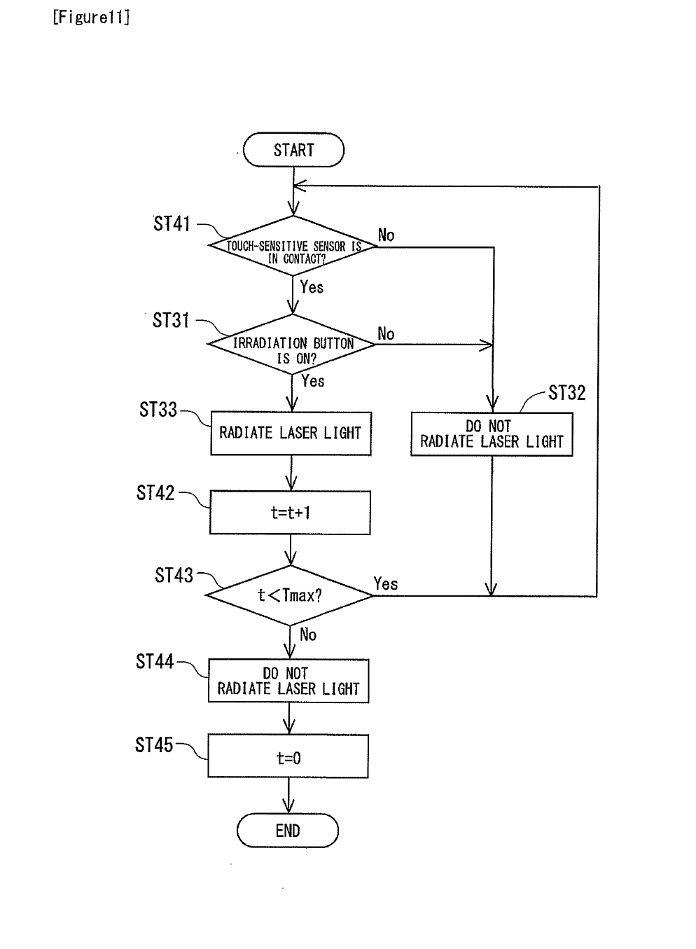

FIG. 10 is a flow chart showing the operation of the laser treatment device according to the fourth embodiment of this invention.

When the power source 71 is turned on to activate the laser treatment device 10, first, the CPU 74 detects if there is contact with the skin, which is irradiated with the laser light, by the touch-sensitive sensor 80 and the touch-sensitive sensor processing circuit 75 (step ST41). If there is no contact (NO in step ST41), the CPU 74 does not carry out irradiation of the laser light (step ST32) in order to prevent a trouble such as erroneous irradiation of laser light to, for example, eyes, and a standby state is obtained (NO in step ST41 and step ST32).

If contact with the skin is detected by the touch-sensitive sensor 80 and the touch-sensitive sensor processing circuit 75 in the standby state (YES in step ST41), the CPU 74 subsequently determines whether the irradiation button 21 is turned on or not (step ST31). While the irradiation button 21 is not turned on (NO in step ST31), the CPU 74 does not cause the laser light to be emitted from the laser light source (step ST32) and becomes the standby state (YES in step ST41, NO in step ST31, and step ST32).

Then, if contact with the skin is detected (YES in step ST41) and if it is detected that the irradiation button 21 has been turned on (YES in step ST31), first time in this process, the CPU 74 controls the laser power control circuit 72 and causes the laser light to be emitted from the laser light source 40 (step ST33). The laser light is radiated to the skin through the light conductor 50 and the light diffusion plate 51.

In this manner, the CPU 74 detects contact/non-contact with the skin, which is to be irradiated with the laser light, by using the touch-sensitive sensor 80 and the touch-sensitive sensor processing circuit 75. Then, in the case of non-contact, irradiation of the laser light is not carried out from the viewpoint of preventing erroneous radiation. On the other hand, in the case of contact, on the condition that the irradiation button 21 is turned on, the CPU 74 controls the laser power control circuit 72 and carries out irradiation of the laser light As a result, erroneous radiation with respect to part other than the irradiation part, particularly, eyes can be prevented, and the user can be protected from unexpected troubles.

As explained below, a limit may be provided on the irradiation time of the laser light.

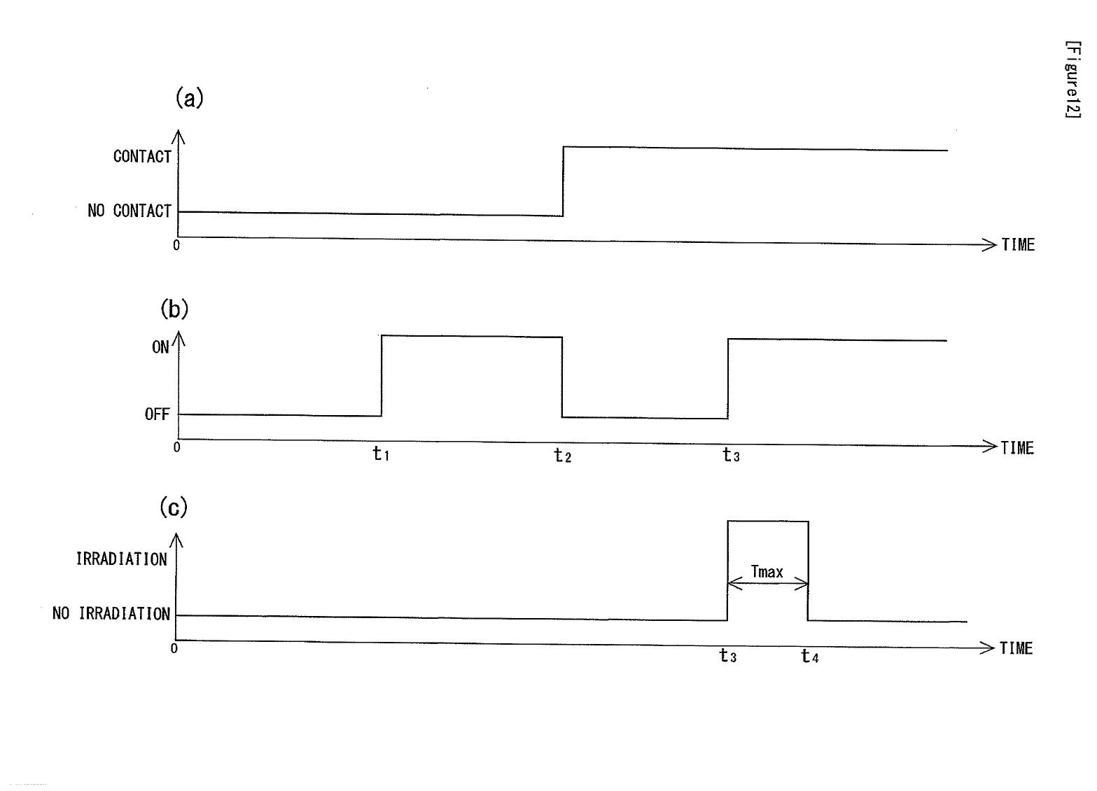

FIG. 11 is a flow chart of the operation of the laser treatment device according to the fourth embodiment of this invention. Since step ST41 and steps ST31 to ST33 of FIG. 11 are operations similar to those of FIG. 10, explanation thereof will be omitted, and step ST42 and thereafter will be explained below.

When laser light is radiated from the laser light source 40 in step ST33, the CPU 74 activates a timer in order to measure irradiation time t of the laser light and increments the irradiation time t by unit irradiation time (step ST42).

Then, the CPU 74 compares the irradiation time t with maximum irradiation time Tmax and determines whether the irradiation time t has reached the maximum irradiation time Tmax or not (step ST43). if the irradiation time t is less than the maximum irradiation time Tmax (YES in step ST43), as long as the both conditions of the touch-sensitive sensor 80 and the irradiation button 21 are satisfied (YES in step ST41, YES in step ST31), the CPU 74 repeats irradiation of the laser light (step ST33), increment of the irradiation time t (step ST42), and comparison determination of the irradiation time (step ST43).

In this manner, the laser light is radiated to continue increasing the irradiation time t. If the time reaches the maximum irradiation time Tmax (NO in step ST43), the CPU 74 determines that the irradiation of the laser light with respect to the skin of a single location exceeding the maximum irradiation time Tmax is excessive irradiation, controls the laser power control circuit 72 in order to suppress influence on the skin, and forcibly stops the irradiation of the laser light (step ST44). Then, the timer is reset for irradiation of next time (step ST45), and the series of processes is finished. Thereafter, a new process for another irradiation part is newly carried out from step ST41.

FIG. 12 shows timing charts for explaining the operation of the laser treatment device of FIG. 11. FIG. 12 (a) shows contact/non-contact of the touch-sensitive sensor 80, FIG. 12 (b) shows ON/OFF of the irradiation button 21, and FIG. 12 (c) shows irradiation/no-irradiation of the laser light.

In time [0 to t1], time [t1 to t2], time [t2 to t3], the CPU 74 does not cause the laser light to be emitted from the laser light source 40 since the touch-sensitive sensor 80 is not detecting contact with the skin or the irradiation button 21 is OFF. At the time t3 and thereafter when the touch sensor 80 detects the contact and the irradiation button 21 is ON to satisfy both the conditions, the CPU 74 radiates the laser light to the skin for the maximum irradiation time Tmax as shown in FIG. 12 (c). Then, at time t4 and thereafter, the CPU 74 stops irradiation of the laser light regardless of the both of the conditions of the contact of the touch-sensitive sensor 80 and ON of the irradiation button 21.

In this manner, even if the touch-sensitive sensor 80 continues contacting the skin of a single location and the irradiation button 21 continues being pressed, irradiation of the laser light is stopped after the skin of the single location continues being irradiated with the laser light for the maximum irradiation time Tmax. As a result, excessive irradiation of the laser light with respect to the skin of the single location is prevented so as to ensure safety of the user.

As the value of the maximum irradiation time Tmax, for example, the maximum irradiation time Tmax may be stored in advance in an unshown memory so that the CPU 74 reads the maximum irradiation time Tmax from the memory when the process of step ST43 is to be carried out; alternatively, the maximum irradiation time Tmax may be determined with reference to the value of the reflected light power P1 obtained by the color sensor 60 shown in the third embodiment.

As described above, according to the fourth embodiment, the device is provided with: the touch-sensitive sensor 80 and the touch-sensitive sensor processing circuit 75, which detect in step ST41 the contact with the skin to be irradiated with the laser light; and the CPU 74 which controls the laser power control circuit 72 in step ST33 in accordance with ON of the irradiation button 21 and causes the laser light source 40 to emit laser light only while the touch-sensitive sensor 80 and the touch-sensitive sensor processing circuit 75 detect contact with the skin to obtain YES in step ST41. Therefore, as long as contact with the skin to be irradiated with the laser light is not detected by the touch-sensitive sensor 80 and the touch-sensitive sensor processing circuit 75, the laser light source 40 does not emit laser light, an unexpected trouble that, for example, eyes are erroneously irradiated with laser light can be prevented, and an effect that safety of the high-power laser treatment device 10 can be ensured is obtained.

Moreover, according to the fourth embodiment, after the laser light is radiated to the skin for the predetermined maximum irradiation time Tmax and NO is obtained in step ST43, the CPU 74 controls the laser power control circuit 72 so as to cause the laser light source 40 to stop emission of the laser light in step ST44. Therefore, the trouble that laser light is excessively radiated to the skin of the single location can be prevented, and an effect that safety of the high-power laser treatment device 10 can be further ensured is obtained.

### REFERENCE SIGNS LIST

10 Laser treatment device, 20 Gripping part, 21 Irradiation button, 30 Head unit, 30A Opening, 40 Laser light source (light source means), 41 VCSEL array, 41s VCSEL element, 42 Microlens array, 50 Light guide (light guiding means), 51 Light diffusion plate (light diffusion means), 60 Color sensor (reflected-light power detection means), 70 Circuit, 71 Power source, 72 Laser power control circuit (light source means), 73 Color sensor processing circuit (reflected-light power detection means), 74 CPU (control means), 75 Touch-sensitive sensor processing circuit (contact detection means), 80 Touch-sensitive sensor (contact detection means), L Laser light, and D Synthetic intensity distribution.
1 FIG. 1
2 VCSEL ARRAY
3 IRRADIATION BUTTON
4 POWER SOURCE
5 LASER POWER CONTROL CIRCUIT
6 COLOR SENSOR
7 LASER LIGHT SOURCE
8 IRRADIATION BUTTON IS ON?
9 RADIATE LASER LIGHT
10 DO NOT RADIATE LASER LIGHT
11 DETECT REFLECTED LIGHT POWER P₁
12 INCREASE THE POWER OF LASER LIGHT EMITTED FROM LASER LIGHT SOURCE (MAINTAIN IF P₁=P₀)
13 DECREASE THE POWER OF LASER LIGHT EMITTED FROM LASER LIGHT SOURCE
14 TOUCH-SENSITIVE SENSOR PROCESSING CIRCUIT
15 TOUCH-SENSITIVE SENSOR
16 TOUCH-SENSITIVE SENSOR IS IN CONTACT?
17 CONTACT NO CONTACT
18 IRRADIATION NO IRRADIATION
19 TIME

1. A laser treatment device comprising
a light source means provided with at least one or more surface-emitting laser array that has two or more laser elements arranged on a single wafer and emits laser light for irradiating an irradiation part;
a drive power source that drives the light source means;
a light diffusion means that diffuses the laser light for irradiating the irradiation part;
a reflected-light power detection means that detects power of reflected light of the light irradiating the irradiation part, the reflected light reflected from the irradiation part; and
a control means that adjusts, in accordance with the power of the reflected light detected by the reflected-light power detection means, power of the laser light emitted from the light source means; wherein
the reflected-light power detection means is provided near an emitting side of the light diffusion means.

2. The laser treatment device according to claim 1, wherein
the light source means uses the two surface-emitting laser arrays having a drive voltage of 2 V; and
the drive power source is four rechargeable batteries connected in series, wherein each rechargeable battery has a voltage of 1.2 V.

3. The laser treatment device according to claim 1, wherein
the light source means is provided with a light guide means that receives the emitted laser light and guides the light to the irradiation part.

4. (canceled)

5. (canceled)

6. (canceled)

7. (canceled)

8. The laser treatment device according to claim 1 comprising
a contact detection means that detects contact with the irradiation part to be irradiated with the laser light; and
a control means that causes the light source means to emit laser light only while the contact detection means is detecting the contact with the irradiation part.

9. The laser treatment device according to claim 8, wherein,
after the irradiation part is irradiated with the laser light for predetermined time, the control means causes the light source means to stop emission of the laser light.
